(19) United States
(12) Patent Application Publication
(10) Pub. No.: US 2018/0368786 A1
(43) Pub. Date: Dec. 27, 2018

(54) THREE-DIMENSIONAL SCATTERED RADIATION IMAGING APPARATUS, RADIOLOGICAL MEDICAL SYSTEM HAVING THE SAME, AND METHOD FOR ARRANGING THREE-DIMENSIONAL SCATTERED RADIATION IMAGING APPARATUS

(71) Applicant: KOREA UNIVERSITY RESEARCH AND BUSINESS FOUNDATION, Seoul (KR)

(72) Inventors: Won Ho LEE, Seoul (KR); Tae Woong LEE, Seoul (KR)

(73) Assignee: KOREA UNIVERSITY RESEARCH AND BUSINESS FOUNDATION, Seoul (KR)

(21) Appl. No.: 16/063,469

(22) PCT Filed: Dec. 7, 2016
(86) PCT No.: PCT/KR2016/014278

§ 371 Date: Jun. 18, 2018

(30) Foreign Application Priority Data
Dec. 17, 2015 (KR) 10-2015-0180745
Nov. 18, 2016 (KR) 10-2016-0154335

(51) Int. Cl. A61B 6/03; A61B 6/00; G06T 7/00; G01T 1/166

(52) U.S. Cl. CPC A61B 6/035; A61B 6/5282; G01T 1/1663; G06T 7/0012; A61B 6/483

(57) ABSTRACT

The three-dimensional scattered radiation imaging apparatus of the present invention includes: a detection unit which includes a first detector for detecting the position and energy of radiation irradiated from a radiation source and scattered from a subject, a second detector for detecting the position and energy of radiation scattered from the first detector, and a third detector for detecting the position and energy of radiation scattered from the second detector; a signal processing unit for receiving, from the first detector, the second detector, and the third detector of the detection unit, information on the positions and energy of the radiation detected by the first detector, the second detector, and the third detector of the detection unit; and an image processing unit for receiving information from the signal processing unit and displaying the information as an image.

### TECHNICAL FIELD

The present invention relates to a radiation imaging apparatus. More specifically, the present invention relates to a three-dimensional scattered radiation imaging apparatus which can reconstruct, as an image, information on radiation scattered from the human body and regarded as a noise during radiotherapy and determine the irradiation position and the dose distribution of radiation using the reconstructed image, a radiological medical system having the same, and a method for arranging the three-dimensional scattered radiation imaging apparatus.

### BACKGROUND ART

In recent years, the importance of the advanced medical device industry is increasing along with the improvement in quality of life. In the medical device industry, a medical imaging diagnostic apparatus can be regarded as a part of a representative cutting-edge high added industry, which accounts for more than 70% of the entire medical device market.

A medical imaging diagnostic apparatus technology is a collective term for a technology that extracts, processes, analyzes, manages, and outputs data essential for diagnosis and treatment of diseases by quantitatively imaging information on the structure, function, metabolism, and components of the human body's organs, tissues, cells, and molecules. A high-resolution in vivo imaging technique according to the latest technological development is a convergence technology including IT, BT and NT. This is one of the next-generation core technologies, which enables the diagnosis of diseases making it difficult to precisely diagnose with a conventional technology as well as the early diagnosis of diseases before the full expression of the diseases.

Examples of the medical imaging diagnostic apparatus include an X-ray photographing apparatus, a computed tomograph (CT) apparatus, a magnetic resonance imaging (MRI) apparatus, a diagnostic ultrasound scanner, a positron emission tomography (PET) apparatus, and the like. Specifically, examples of a radioactive imaging diagnostic apparatus include an X-ray photographing apparatus, a computed tomograph (CT) apparatus, and a positron emission tomography (PET) apparatus.

Such a medical imaging diagnostic apparatus may display tumors non-invasively and makes it possible to help physician's access to examine the presence or absence of invasion and the size of surrounding tissues. As such, an image-based examination occupies a very important position that is essential in modern medical diagnostics. In addition, the advancement of the computer hardware and software technology that enables a high-speed operation makes it possible to diagnose diseases using a three-dimensional image rather than a simple two-dimensional image. Recently, a medical imaging diagnostic technology follows a trend toward reduction in the photographing time, high resolution, and three-dimensionalization in a cross section of the medical imaging diagnostic industry, and the development of various diagnostic analysis methods is in progress.

In the meantime, radiation therapy generally employs a method in which the irradiation position and the dose distribution of radiation irradiated are computationally predicted in a radiation irradiation planning stage, or in which water or a phantom similar to the human body is disposed and a glass dosimeter or an ion chamber is put therein to measure experimental values.

However, such predictable computer simulation or preliminary experiment may differ from an actual radiation irradiation environment, which results in uncertainty. In addition, there is a limitation in the accuracy of prediction due to a change in the measured value according to actual irradiation despite a similar environment. In a radiation measurement method, either film or EPID is positioned and used on the opposite side of a radiation incident direction relative to a patient, but the resolution of the image is degraded and the film or EPID should be located on a line extending in the radiation irradiation direction. For this reason, an image obtained by such a conventional radiation measurement method is limited to a two-dimensional image, so that even when a three-dimensional image is to be acquired, it can be predicted only computationally.

### DISCLOSURE OF INVENTION

### Technical Problem

The present invention has been made to solve the above-mentioned problems associated with the prior art, and it is an object of the present invention to provide a three-dimensional scattered radiation imaging apparatus in which a detector for detection of radiation is arranged three-dimensionally or combined with a radiological medical system to reconstruct, as an image, information on radiation irradiated to and then scattered from the human body so that the irradiation position and the dose distribution can be three-dimensionally measured in real time, the radiological medical system including the same, and a method for arranging the three-dimensional scattered radiation imaging apparatus.

### Technical Solution

To achieve the above objects, in one aspect, the present invention provides a three-dimensional scattered radiation imaging apparatus including: a detection unit which includes a first detector for detecting the position and energy of radiation irradiated from a radiation source and scattered from a subject, a second detector for detecting the position and energy of radiation scattered from the first detector, and a third detector for detecting the position and energy of radiation scattered from the second detector; a signal processing unit for receiving, from the first detector, the second detector, and the third detector of the detection unit, information on the positions and energy of the radiation detected by the first detector, the second detector, and the third detector of the detection unit so as to obtain the position of the radiation source in such a manner as to reversely track the incident direction of the radiation; and an image processing unit for receiving information from the signal processing unit and displaying the information as an image.

In the three-dimensional scattered radiation imaging apparatus of the present invention, the detection unit may be implemented as a Compton camera structure in which each of the first detector, the second detector, and the third detector includes a scintillator and an optical sensor.

In the three-dimensional scattered radiation imaging apparatus of the present invention, the detection unit is implemented as a Compton camera structure in which each of the first detector, the second detector, and the third detector includes a semiconductor material selected from among CdTe, CZT, and T1Br.

In the three-dimensional scattered radiation imaging apparatus of the present invention, the signal processing unit may calculate an energy (E) expressed as the following equation (3) through the following equations (1) and (2) when the direction of radiation irradiated from the radiation source to the subject is known, the energy (E) being absorbed by the subject:

$${hv}^{′} = \frac{hv}{1 + \frac{hv}{{m}_{0} {C}^{2}} ( 1 - cos θ )} \qquad ( 1 )$$

wherein hv′ denotes the energy of a photon scattered from the subject, hv denotes the energy of a photon irradiated from the radiation source, θ denotes the scattered angle of radiation scattered from the subject, and m₀c² denotes the rest mass of an electron,

$$\begin{matrix}E = hv - {hv}^{′} & ( 2 ) \\ E = hv - {hv}^{′} = {hv}^{′} \frac{\frac{{hv}^{′}}{{m}_{0} {C}^{2}} ( 1 - cos θ )}{1 - \frac{{hv}^{′}}{{m}_{0} {C}^{2}} ( 1 - cos θ )} . & ( 3 )\end{matrix}$$

In the three-dimensional scattered radiation imaging apparatus of the present invention, the signal processing unit may detect the energy and direction of radiation scattered from the subject and incident on the first, second and third detectors when the direction of radiation irradiated from the radiation source to the subject is not known, and calculate the dose of radiation irradiated to the subject by comparing a theoretical value and a computer simulation value/an actual measurement value.

In the three-dimensional scattered radiation imaging apparatus of the present invention, the signal processing unit may three-dimensionally represent the positions of radiation scattered from the first, second and third detectors, and represent them in a four-dimensional matrix, including the energy absorbed by the subject so as to calculate the energy absorbed by the subject.

In the three-dimensional scattered radiation imaging apparatus of the present invention, the signal processing unit may calibrate an absolute value for the energy absorbed by the subject through a simulation that is performed before the radiation irradiation when the angle of radiation incident on the subject is not known.

In the three-dimensional scattered radiation imaging apparatus of the present invention, the detection unit may be arranged in a direction where the degree of uncertainty of detection is low to fit the incident energy-dependent scattering distributions of radiation based on the Klein-Nishina formula depending on the energy absorbed by the subject and the energy scattered from the subject.

To achieve the above objects, in another aspect, the present invention provides a radiation medical system including: a radiation irradiation unit for irradiating a subject with radiation; a radiation detection unit comprising a detection unit for detecting the position and energy of radiation irradiated from the radiation irradiation unit and scattered from a subject, a signal processing unit for receiving information on the positions and energy of the radiation detected by the detection unit from the detection unit so as to obtain the position of the radiation irradiation unit in such a manner as to reversely track the incident direction of the radiation, and an image processing unit for receiving information from the signal processing unit and displaying the information as an image; and a controller for controlling the radiation irradiation unit and the radiation detection unit.

The radiological medical system of the present invention may further include a driver for movably operating the radiation irradiation unit, and the radiation detection unit may be coupled with the radiation irradiation unit so as to detect radiation scattered from the subject while being movably operated together with the radiation irradiation unit by the driver.

The radiological medical system of the present invention may further include an irradiation unit driver for movably operating the radiation irradiation unit; and a detection unit driver for movably operating the radiation detection unit.

In the radiological medical system of the present invention, the radiation detection unit may be provided in plural numbers so as to be spaced apart from one another.

In the radiological medical system of the present invention, the detection unit of the radiation detection unit may include: a first detector for detecting the position and energy of radiation irradiated from the radiation irradiation unitand scattered from the subject; a second detector for detecting the position and energy of radiation scattered from the first detector; and a third detector for detecting the position and energy of radiation scattered from the second detector.

In the radiological medical system of the present invention, the radiation detection unit may include a Compton camera structure in which the detection unit comprises a scintillator and an optical sensor.

In the radiological medical system of the present invention, the radiation detection unit may include a Compton camera structure in which the detection unit comprises a semiconductor material selected from among CdTe, CZT, and T1Br.

In the radiological medical system of the present invention, the radiation detection unit may further include a collimator for collimating radiation scattered from the subject and applying the collimated radiation to the detection unit.

In the radiological medical system of the present invention, the radiation detection unit may further include a CT detector coupled with the radiation detection unit for reconstructing a three-dimensional image.

In the radiological medical system of the present invention, the detection unit of the radiation detection unit may be arranged in a direction where the degree of uncertainty of detection is low to fit the incident energy-dependent scattering distributions of radiation based on the Klein-Nishina formula depending on the energy E absorbed by the subject and the energy scattered from the subject.

To achieve the above objects, in still another aspect, the present invention provides a method for arranging a three-dimensional scattered radiation imaging apparatus, comprising the steps of: (a) provisionally arranging the three-dimensional scattered radiation imaging apparatus comprising a detection unit of a Compton camera structure for detecting the position and energy of radiation irradiated from a radiation source and scattered from a subject, a signal processing unit for receiving information on the positions and energy of the radiation detected by the detection unit from the detection unit so as to obtain the position of the radiation source in such a manner as to reversely track the incident direction of the radiation, and an image processing unit for receiving information from the signal processing unit and displaying the information as an image; (b) calculating an energy E expressed as the following equation (3) through the following equations (1) and (2) using the detection unit of the three-dimensional scattered radiation imaging apparatus, the energy E being absorbed by the subject

$${hv}^{′} = \frac{hv}{1 + \frac{hv}{{m}_{0} {C}^{2}} ( 1 - cos θ )} \qquad ( 1 )$$

wherein hv′ denotes the energy of a photon scattered from the subject, hv denotes the energy of a photon irradiated from the radiation source, θ denotes the scattered angle of radiation scattered from the subject, and m₀c² denotes the rest mass of an electron,

$$\begin{matrix}E = hv - {hv}^{′} & ( 2 ) \\ E = hv - {hv}^{′} = {hv}^{′} \frac{\frac{{hv}^{′}}{{m}_{0} {C}^{2}} ( 1 - cos θ )}{1 - \frac{{hv}^{′}}{{m}_{0} {C}^{2}} ( 1 - cos θ )} ; & ( 3 )\end{matrix}$$

(c)_calculating an average value of the energy-dependent ratios of a total attenuation coefficient (the probability of reaction and attenuation of radiation per unit length) and a scattering coefficient (the probability of Compton scattering of radiation per unit length) of radiation based on the amount of incident radiation at the energy, and calibrating the energy E of the photon absorbed by the subject and the energy of the photon scattered from the subject, which are calculated in step (b) based on the averaged ratios; and (d) adjusting the position of the three-dimensional scattered radiation imaging apparatus in a direction where the degree of uncertainty of detection is low to fit the incident energy-dependent scattering distributions of radiation based on the Klein-Nishina formula depending on the energy E absorbed by the subject and the energy scattered from the subject, which are calibrated in step (c).

In the method for arranging a three-dimensional scattered radiation imaging apparatus of the present invention, steps (b) to (d) may be performed repeatedly in order to further optimize the arrangement of the detection unit of the three-dimensional scattered radiation imaging apparatus.

### Advantageous Effects

The radiological medical system including the three-dimensional scattered radiation imaging apparatus according to this embodiment as constructed above can, in real time, measure radiation during the radiotherapy beyond the prediction using a conventional specimen or computer simulation, and obtain a three-dimensional distribution of radiation irradiated beyond a conventional plane distribution. Furthermore, a result can be obtained without being accompanied by an additional dose examination, and thus the present invention can be applied to an existing radiological medical system to enable more improved treatment observation.

In addition, the three-dimensional scattered radiation imaging apparatus according to the present invention is installed in such a manner as to be combined with the radiological medical system so that the irradiation position and the dose distribution of radiation can be measured in real time by a detection method employing a multi-scattering technique during radiotherapy using the radiological medical system.

Further, the use of the arrangement method of the three-dimensional scattered radiation imaging apparatus of the present invention can arrange the detection unit of the three-dimensional scattered radiation imaging apparatus in such a manner as to obtain the maximum detection efficiency and the most effective detection information.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a schematic diagram showing a radiological medical system having a three-dimensional scattered radiation imaging apparatus according to an embodiment of the present invention;

FIG. 2 is a block diagram showing a main configuration of the radiological medical system shown in FIG. 1;

FIG. 3 is a schematic block diagram showing a configuration of the three-dimensional scattered radiation imaging apparatus shown in FIG. 1;

FIG. 4 is a schematic view showing the Compton scattering of radiation;

FIG. 5 is a diagrammatic view showing a method for detecting the position of a radiation source using the three-dimensional scattered radiation imaging apparatus shown in FIG. 1;

FIG. 6 is a flow chart showing a process of arranging the three-dimensional scattered radiation imaging apparatus shown in FIG. 1;

FIG. 7 shows incident energy-dependent scattering distributions based on the Klein-Nishina formula in the Compton scattering of radiation;

FIGS. 8 to 10 are schematic block diagrams showing various modifications of a three-dimensional scattered radiation imaging apparatus according to the present invention; and

FIGS. 11 and 12 are schematic diagrams showing various modifications of a radiological medical system having a three-dimensional scattered radiation imaging apparatus according to the present invention.

### BEST MODE FOR CARRYING OUT THE INVENTION

Now, a three-dimensional scattered radiation imaging apparatus and a radiological medical system having the same according to the preferred embodiments of the present invention will be described hereinafter in detail with reference to the accompanying drawings. It should be noted that the same elements in the drawings are denoted by the same reference numerals although shown in different figures. In the following description, the detailed description on known function and constructions unnecessarily obscuring the subject matter of the present invention will be avoided hereinafter.

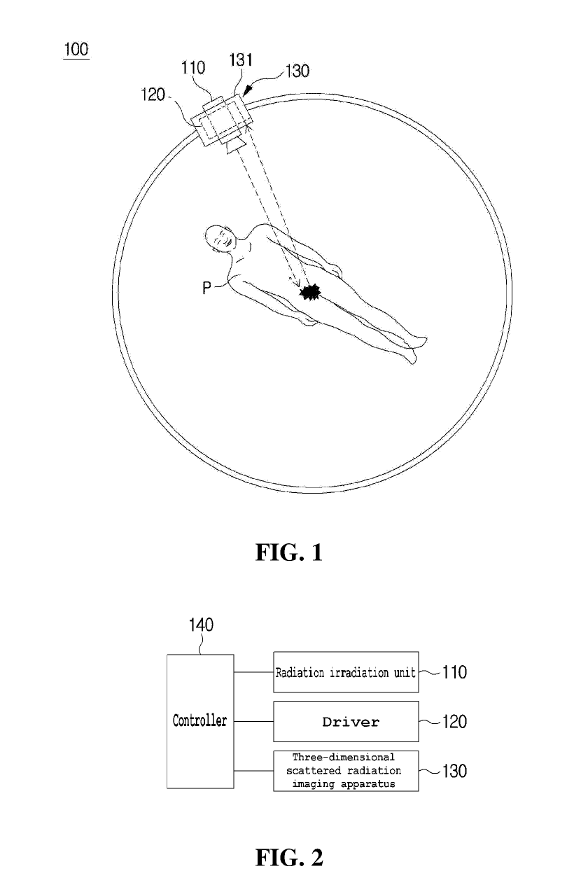

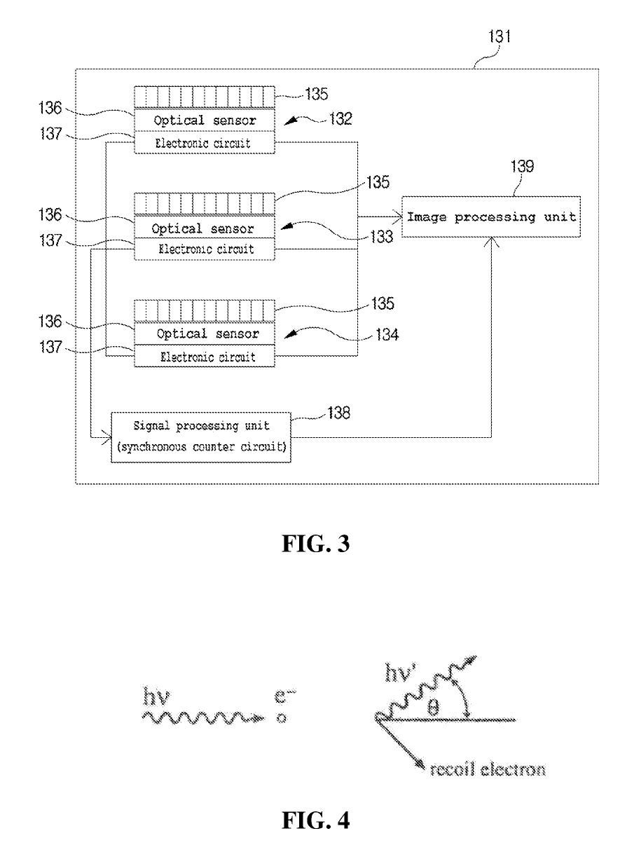

FIG. 1 is a schematic diagram showing a radiological medical system having a three-dimensional scattered radiation imaging apparatus according to an embodiment of the present invention, FIG. 2 is a block diagram showing a main configuration of the radiological medical system shown in FIG. 1, and FIG. 3 is a schematic block diagram showing a configuration of the three-dimensional scattered radiation imaging apparatus shown in FIG. 1.

As shown in FIGS. 1 to 3, the radiological medical system 100 having a three-dimensional scattered radiation imaging apparatus according to an embodiment of the present invention includes a radiation irradiation unit 110 that irradiates a subject with radiation, a three-dimensional scattered radiation imaging apparatus 130 that detects the irradiation position and the dose distribution of radiation, and a controller 140 that controls the radiation irradiation unit 110 and the three-dimensional scattered radiation imaging apparatus 130. The radiological medical system 100 according to the present invention includes the three-dimensional scattered radiation imaging apparatus 130 that can more precisely detect the irradiation position and the dose distribution of radiation in real time so as to solve problems in that the real-time measurement of the irradiation position and the dose distribution of radiation of a conventional prediction method is impossible and in that the irradiation direction of radiation and the direction of the detector are necessarily required to be identical to each other in a real-time measurement method so that an error resulting from uncertainty in an actual radiotherapy environment can be avoided. The radiological medical system 100 according to the present invention can be applied to various radiological medical systems such as the LINAC or tomotherapy system.

The radiation irradiation unit 110 is movably operated by a driver so as to irradiate a patient P with radiation while rotating 360 degrees around the patient P. The radiation irradiation unit 110 and the driver 120 are controlled by a controller 140.

The three-dimensional scattered radiation imaging apparatus 130 is integrally coupled with the radiation irradiation unit 110. The three-dimensional scattered radiation imaging apparatus 130 is movably operated together with the radiation irradiation unit 110 by the driver 120 so as to, in real-time, measure radiation scattered from the patient P while rotating 360 degrees around the patient P.

The three-dimensional scattered radiation imaging apparatus 130 includes a radiation detection unit 131. The radiation detection unit 131 includes a plurality of detectors 132, 133 and 134, a signal processing unit 138, and an image processing unit 139. The plurality of detectors 132, 133 and 134 constitute a detection unit 131 a implemented as a Compton camera structure that detects radiation by an electronic collimation method.

As known in the art, the Compton camera is a radiation imaging apparatus that images a three-dimensional distribution of a radiation source using the Compton scattering principle. The Compton camera includes a detector of a scatterer and an absorber, and acquires the direction information of an incident photon using information on the energy and the detection position measured from the scatterer and the absorber. In other words, a path along which the photon is subjected to Compton scattering and then proceeds to the absorber can be grasped from the detection position of the scatterer and the absorber, and an axis of connecting this path can be created. In addition, it can be seen that because the scattering angle could be calculated from the energy measured by the scatterer, the photon was incident at the scattering angle about the created angle. However, it can be presumed that because the incident angle of the photon cannot be grasped, the photon was emitted from any one point on an elliptical cone surface having a half angle of the scattering angle. But, in principle, if there are three elliptical cones, it is possible to infer an original position where the photon was emitted on a three-dimensional space by finding a crossing point of these elliptical cones. This Compton camera employs an electronic collimation method which presumes the position of a radiation source using only information on the detection position and the energy measured by the detector without any mechanical collimation devices so that various limitations of conventional radiation imaging apparatuses for nuclear medicine imaging, nondestructive testing, and cosmic radiation measurement can be overcome.

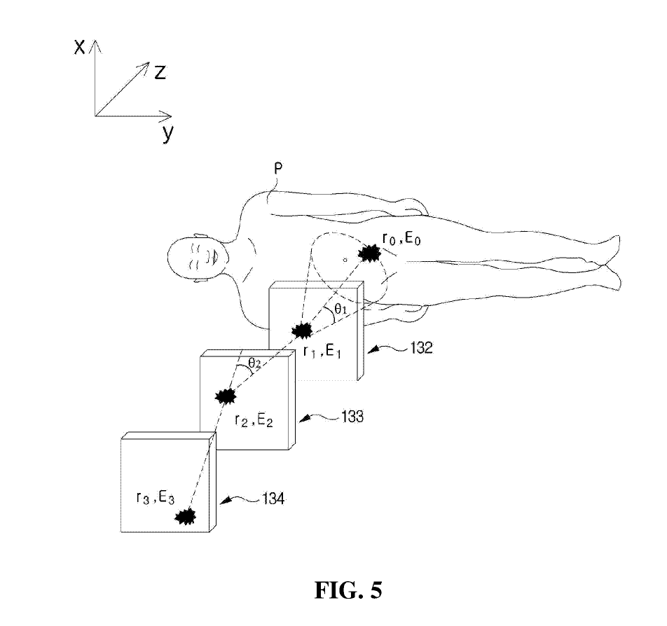

Referring FIGS. 3 and 5, the plurality of detectors 132, 133 and 134 constituting the detection unit 131a of a Compton camera structure are arranged to be spaced apart from one another. The detectors 132, 133 and 134 have the same structure, and detect the position and energy of radiation scattered from a radiation irradiated subject such as the patient P. Each of the plurality of detectors 132, 133 and 134 includes a scintillator 135, an optical sensor 136, and an electronic circuit 137. Herein, PSPMT, SiPM or the like can be used as the optical sensor 136. The first detector 132 detects the position and energy of radiation irradiated from the radiation irradiation unit 110 as a radiation source and scattered from the patient P. The second detector 133 detects the position and energy of radiation having passed through the first detector 132, and the third detector 134 detects the position and energy of radiation having passed through the second detector 133. The signal processing unit 138 receives information on the positions and energy of the radiation from the plurality of detectors 132, 133 and 134 so as to calculate the position of the radiation source in such a manner as to reversely track the incident direction of the radiation. The image processing unit 139 receives information from the signal processing unit 138 and displays the information as an image.

The three-dimensional scattered radiation imaging apparatus 130 including the radiation detection unit 131 of the Compton camera structure allows the radiation to proceed while efficient Compton scattering continuously occurs in the first detector 132 and the second detector 133 of the radiation detection unit 131, and allows the radiation to be photoelectrically absorbed or the Compton scattering to occur in the third detector 134. In this case, the first to third detectors 132, 133 and 134 acquire the position information and the energy information of the incident radiation, and transmit the acquired information to the signal processing unit 138. The signal processing unit 138 acquires information on the position and kind of the radiation source based on various items of information applied thereto from the detectors, and the image processing unit 139 receives information from the signal processing unit 138 and implements the received information as an image.

The three-dimensional scattered radiation imaging apparatus 130 of the radiological medical system according to this embodiment can calculate the position of a radiation source by different methods for both the cases where the direction of radiation irradiated from the radiation irradiation unit 110 is known and where it is not known.

First, the case where the direction of radiation irradiated from the radiation irradiation unit 110 is known will be described with reference to FIGS. 1 to 4. FIG. 4 is a schematic view showing the Compton scattering of radiation. When the direction of radiation irradiated from the radiation irradiation unit 110 and incident on a patient P and the arrangement angle of the detectors 132, 133 and 134 of the radiation detection unit 131 are known, the scattered angle 6 of the radiation and the energy hv′ absorbed by the radiation detection unit 131 can be grasped. Thus, the original energy hv of radiation can be calculated using the following equation (1):

$${hv}^{′} = \frac{hv}{1 + \frac{hv}{{m}_{0} {C}^{2}} ( 1 - cos θ )} \qquad ( 1 )$$

wherein e-denotes an electron inside a material, hv denotes the energy of an incident photon, hv′ denotes the energy of a scattered photon, θ denotes the scattered angle of the photon, and m₀c² denotes the rest mass of an electron. Thus, when the energy hv′ absorbed by the radiation detection unit 131 and the original energy hv are grasped, the energy E absorbed by the patient P can be derived from the following equation (2):

E=hv−hv′ (2).

An equation obtained by representing the scattered angle of the photon in the equation (1) as the energy of the incident photon and the energy of the scattered photon can be expressed as follows:

$$cos θ = 1 - ( \frac{{m}_{0} {C}^{2}}{hv} \cdot \frac{{hv}^{′}}{hv - {hv}^{′}} ) .$$

If the scattered angle is substituted into the Equation 2, the following final energy-related equation can be derived:

$$E = hv - {hv}^{′} = {hv}^{′} \frac{\frac{{hv}^{′}}{{m}_{0} {C}^{2}} ( 1 - cos θ )}{1 - \frac{{hv}^{′}}{{m}_{0} {C}^{2}} ( 1 - cos θ )} . \qquad ( 3 )$$

The equation (3) is an equation in which the equation (1) and the equation (2) are added so that the energy absorbed by the patient P is represented as the energy and the scattered angle of the scattered photon.

When the direction of radiation irradiated from the radiation irradiation unit 110 to the patient P is not known, the signal processing unit can detect the energy and direction of radiation scattered from the patient P and incident on the radiation detection unit 131 and calculate the dose of radiation irradiated to the patient P by comparing a theoretical value and a computer simulation value/an actual measurement value.

In the meantime, the three-dimensional scattered radiation imaging apparatus 130 of the radiological medical system according to this embodiment can measure the energy of radiation incident on the radiation detection unit 131 even without the entire absorption of radiation scattered from the patient P, and a detailed method thereof will be described below with reference to FIG. 5.

In the arrangement of the detectors 132, 133 and 134 of the radiation detection unit 131 as shown in FIG. 5, the following equations (4) to (7) can be obtained:

$$\begin{matrix}cos {θ}_{1} = 1 - {m}_{0} {C}^{2} ( \frac{1}{{E}_{2}} - \frac{1}{{E}_{1}} ) & ( 4 ) \\ cos {θ}_{2} = 1 - {m}_{0} {C}^{2} ( \frac{1}{{E}_{3}} - \frac{1}{{E}_{2}} ) & ( 5 ) \\ {E}_{2} = \frac{{DE}_{2}}{2} + \frac{1}{2} {( {DE}_{2}^{2} + \frac{4 {m}_{0} {C}^{2} {DE}_{2}}{1 - cos {θ}_{2}} )}^{2} & ( 6 ) \\ {E}_{1} = {DE}_{1} + {E}_{2} = {DE}_{1} + \frac{{DE}_{2}}{2} + \frac{1}{2} {( {DE}_{2}^{2} + \frac{4 {m}_{0} {C}^{2} {DE}_{2}}{1 - cos {θ}_{2}} )}^{2} & ( 7 )\end{matrix}$$

wherein r1, r2 and r3 denote the detection positions of radiation at respective detectors 132, 133 and 134, E1, E2 and E3 denote information on the energy of radiation incident on respective detectors 132, 133 and 134, DE1 and DE2 denote the energies absorbed by the first detector 132 and the second detector 133, θ1 and θ2 denote the scattered angles of radiation, and m₀c² denotes the rest mass of an electron.

The energy E1 of radiation scattered from the patient and incident on the first detector 132 can be calculated even without absorption of the entire energy through the equations (4) to (7).

In the detection of the position of the radiation source, the scattered angle of actual radiation is identified through the scattering position estimated commonly during the detection of a plurality of scattered lines, but not the detection of radiation scattered once. In other words, in the case of a Compton image, a place where actual radiation enters by the formation of one cone (i.e., ring) is one among such cones. Thus, it is determined that radiation enters from a place where overlapped several reactions are commonly designated.

In addition, the positions of radiation scattered from the patient P can be recorded three-dimensionally and can be represented in a four-dimensional matrix, including the energy absorbed by the patient P. If the scattering position estimated commonly as described above is determined, when the angle of radiation incident on the patient is known, the energy absorbed by the patient is calculated. On the contrary, when the angle of radiation incident on the patient is not known, an absolute value for the energy absorbed by the patient is calibrated through a simulation that is performed before the radiotherapy.

As can be seen from the foregoing, the irradiation direction of radiation and the angle and position of the detectors 132, 133 and 134 are very important. Thus, the controller 140 operates the driver 120 while organically connecting the position information of the radiation irradiation unit 110 and the detectors 132, 133 and 134 of the radiation detection unit 131 and transferring and controlling of the position therebetween. In other words, the irradiation of radiation is initiated by a control signal from the controller 140 when the radiation irradiation unit 110 and the radiation detection unit 131 are fixed in the intended positions, at which time, signals acquired are transferred to the controller 140 from the radiation detection unit 131.

More specifically, the radiological medical system is controlled by the following method. First, the driver 120 is operated depending on the position and angle inputted to the controller 140 to position the radiation irradiation unit 110 and the radiation detection unit 131. After the arrangement position of the radiation irradiation unit 110 and the radiation detection unit 131 is identified, an operation control signal from the control 110 is applied to the radiation detection unit 131 and the radiation irradiation unit 110. When a predetermined time lapses, the controller 140 outputs a control signal indicating the stop of radiation irradiation to the radiation irradiation unit 110 and a control signal indicating the stop of signal acquisition to the radiation detection unit 131. The controller 140 performs the processing of the signal acquired from radiation detection unit 131, and controls the driver 120 to be operated to move the radiation irradiation unit 110 and the radiation detection unit 131 to the preset positions.

As described above, the radiological medical system 100 including the three-dimensional scattered radiation imaging apparatus according to this embodiment can reversely track the incident direction of the radiation using information on the detection position and the energy of radiation measured by the first detector 132 and the second detector 133 when radiation scattered from the patient P reacts with and is scattered from the first detector 132 of the three-dimensional scattered radiation imaging apparatus 130, and then is again detected by the second detector 133. In addition, in the case where radiation is scattered from the second detector 133 and is scattered from or absorbed by the third detector 134, the incident direction of the radiation can be reversely tracked using information on the position and energy of the first to third detectors 132, 133 and 134, at which time an image can be acquired even without information on the energy of the scattered radiation. Further, when the energy and direction of radiation scattered from the patient(P) and incident on the radiation detection unit 131 is known, information on the energy of each incident radiations can be grasped, and the dose of radiation irradiated to the patient P can be precisely measured based on the energy information.

In addition, the radiological medical system 100 including the three-dimensional scattered radiation imaging apparatus according to this embodiment can, in real time, measure radiation scattered from the patient P while the three-dimensional scattered radiation imaging apparatus 130 is coupled to the radiation irradiation unit 110 movable by the driver 120 and rotates 360 degrees around the patient P together with the radiation irradiation unit 110 to perform the radiotherapy on the patient P.

Moreover, the radiological medical system 100 including the three-dimensional scattered radiation imaging apparatus according to this embodiment can, in real time, measure radiation during the radiotherapy beyond the prediction using a conventional specimen or computer simulation, and obtain a three-dimensional distribution of radiation irradiated beyond a conventional plane distribution. Furthermore, a result can be obtained without being accompanied by an additional dose examination, and thus the present invention can be applied to an existing radiological medical system to enable more improved radiotherapy observation.

In the meantime, the efficiency of the radiation detection unit 131 constituting the three-dimensional scattered radiation imaging apparatus 130 is closely related with the kind (atom number, density) of a material constituting the detectors 132, 133 and 134 and the distance between the detectors 132, 133 and 134. A spatial resolution of the radiation detection unit 131 largely depends on a position resolution of the detectors 132, 133 and 134 themselves, a ratio with the distance between the detectors 132, 133 and 134, and an energy resolution of the detectors 132, 133 and 134 themselves. The detectors 132, 133 and 134 are arranged in a geometrical structure in which uncertainty is minimized, and the scintillator 135 or the semiconductor material having an excellent energy resolution is used, so that the precise position and the energy distribution of the irradiated radiation can be measured.

A method of arranging the detectors 132, 133 and 134 of the three-dimensional scattered radiation imaging apparatus 130 in a geometrical structure in which uncertainty is minimized will be described hereinafter.

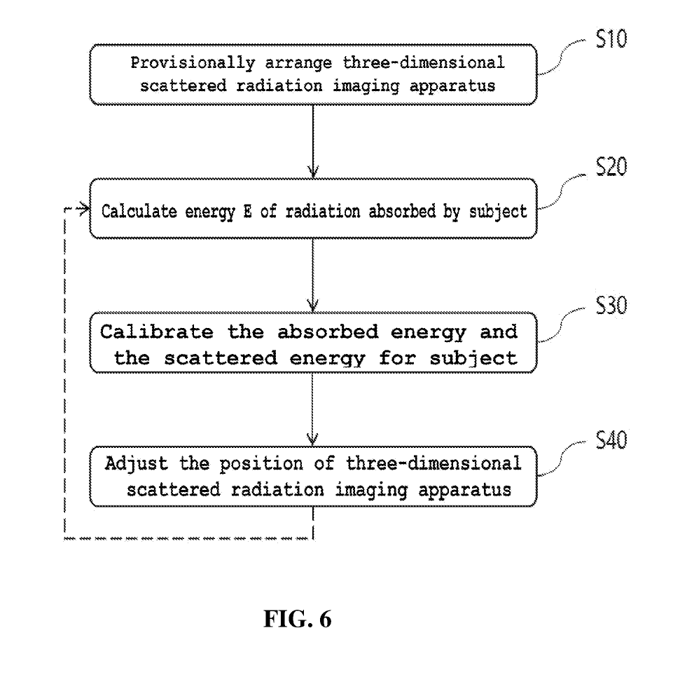

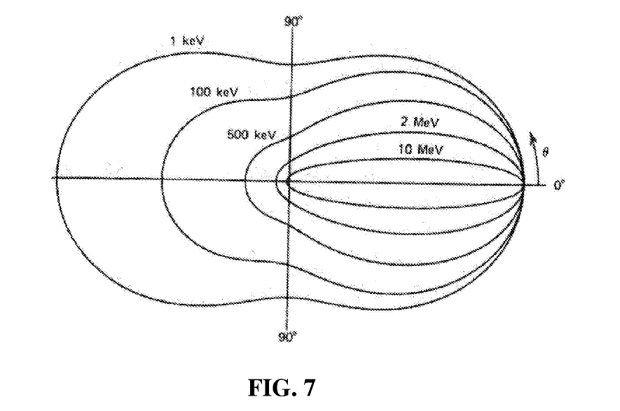

FIG. 6 is a flow chart showing a process of arranging the three-dimensional scattered radiation imaging apparatus shown in FIG. 1.

First, as described above, the three-dimensional scattered radiation imaging apparatus 130 including the detection unit 131a of a Compton camera structure, the signal processing unit 138, and the image processing unit 139 is provisionally arranged at an appropriate position (S10).

Next, the radiation irradiation unit 110 irradiates the subject (e.g., a specimen, a phantom, or the like) with radiation, and the energy E absorbed by the subject is calculated by using the detection unit of the three-dimensional scattered radiation imaging apparatus 130 (S20). The energy E absorbed by the subject can be calculated through the equations (1), (2) and (3).

Subsequently, the controller 140 performs a step (S30) of calibrating the energy absorbed by the subject and the energy scattered from the subject, which are calculated by three-dimensional scattered radiation imaging apparatus 130.

Radiation scattered from the subject, i.e., the human body and incident on the detection unit 131a of the three-dimensional scattered radiation imaging apparatus 130 during radiotherapy is a portion of radiation incident on the human body. The ratio of the actually reacted radiation inside the human body to the scattered radiation varies depending on the constituent material of the detection unit. Thus, if the ration of the incident radiation to the scattered radiation can be grasped through information on the constituent material of the detection unit, the dose of radiation irradiated to the human body can be calculated more precisely.

The constituent material of the detection unit can be predicted through analytical calculation or Monte Carlo computer simulation. A phantom or the like used in the computer simulation may be a phantom proposed in existing KTMAN2 or ORNL. The thus commercialized phantoms are subjected to computer simulation using a program using a Monte Carlo technique. This can calculate the ratio of the scattered radiation to the irradiated radiation. A method of calculating this ratio can be performed by using a method in which the energy-dependent ratios of a total attenuation coefficient (the probability of reaction and attenuation of radiation per unit length) and a scattering coefficient (the probability of Compton scattering of radiation per unit length) of radiation are averaged based on the amount of incident radiation in the energy. Based on the averaged ratios, the actually reacted amount and the scattered amount of radiation at the patient are calibrated so that more excellent results can be obtained. In this case, the energy and amount of the incident radiation can be obtained from a computer simulation before an experiment, a theoretical value on a literature, or an explanatory drawing of a device manufacturer, but a value measured during an actual experiment can be reversely estimated to calculate the energy of the incident radiation, and the energy can be used as a variable to perform a calibration.

The absorbed radiation energy and the scattered radiation energy for the subject can be calibrated by the above method.

Next, the controller 140 performs a step of adjusting the position three-dimensional scattered radiation imaging apparatus 130 (S40). The geometrical arrangement of the detection unit 131a of the three-dimensional scattered radiation imaging apparatus 130 need to be optimized in order to obtain the maximum detection efficiency and the most effective detection information in the detection of radiation scattered from the subject. In other words, the detection unit 131a needs to be positioned in a direction of the scattered radiation in which the detection efficiency is high and the degree of uncertainty of detection information is low.

The Klein-Nishina formula can be used to adjust the position of the detection unit 131a to a position where the detection efficiency is high and the degree of uncertainty of detection is low. Generally, the Compton scattering has different incident energy-dependent scattering distributions based on the Klein-Nishina formula as shown in FIG. 7 (An angle is not a degree but a cosine value). According to this Compton scattering, an optimum efficiency point exists depending on the incident energy. Thus, the position of the detection unit of the three-dimensional scattered radiation imaging apparatus 130 can be adjusted to fit the incident energy-dependent scattering distributions of radiation based on the Klein-Nishina formula depending on the absorbed energy and the scattered energy for the subject as calibrated above.

The use of such a method makes it possible to arrange the detection unit 131a of the three-dimensional scattered radiation imaging apparatus 130 in a direction where the degree of uncertainty of detection is low.

In the method for arranging the three-dimensional scattered radiation imaging apparatus, the above-described steps may be repeatedly performed in order to further optimize the arrangement of the detection unit of the three-dimensional scattered radiation imaging apparatus. In other words, the method for arranging the three-dimensional scattered radiation imaging apparatus uses a regression technique of repeatedly performing a step (S20) of adjusting the position of the three-dimensional scattered radiation imaging apparatus 130 and calculating the energy E of radiation absorbed by the subject at the adjusted position, a step (S30) of calibrating the absorbed energy and the scattered energy for the subject, and a step (S40) of adjusting the position of the three-dimensional scattered radiation imaging apparatus, so that the detection unit 131a of the three-dimensional scattered radiation imaging apparatus 130 can be arranged in such a manner as to obtain the maximum detection efficiency and the most effective detection information.

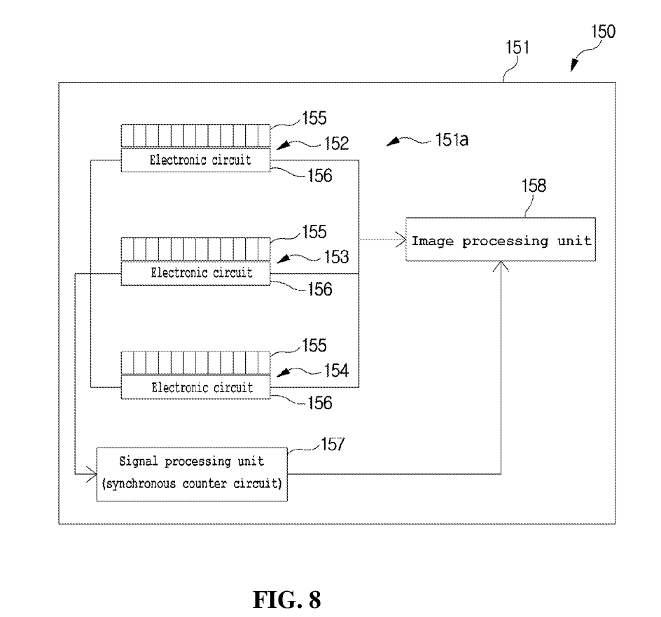

In the meantime, FIGS. 8 to 10 are schematic block diagrams showing various modifications of a three-dimensional scattered radiation imaging apparatus according to the present invention.

As shown in FIG. 8, the three-dimensional scattered radiation imaging apparatus 150 includes a radiation detection unit 151 having a detection unit 131a of a Compton camera structure that detects radiation by an electronic collimation method. The detection unit 151 a includes a plurality of detectors 152, 153 and 154. The radiation detection unit 151 includes the detection unit 151a, a signal processing unit 157, and an image processing unit 158. The plurality of detectors 152, 153 and 154 are disposed to be spaced apart from one another. The detectors 152, 153 and 154 have the same structure, and detect the position and energy of radiation scattered from a radiation irradiated subject such as the patient P. Each of the plurality of detectors 152, 153 and 154 includes a semiconductor material 155 and an electronic circuit 156. The first detector 152 detects the position and energy of radiation irradiated from the radiation irradiation unit 110 (see FIG. 1) as a radiation source and scattered from the patient P. The second detector 153 detects the position and energy of radiation having passed through the first detector 152, and the third detector 154 detects the position and energy of radiation having passed through the second detector 153. The signal processing unit 157 receives information on the positions and energy of the radiation from the plurality of detectors 152, 153 and 154 so as to calculate the position of the radiation source in such a manner as to reversely track the incident direction of the radiation. The image processing unit 158 receives information from the signal processing unit 157 and displays the information as an image.

Herein, the semiconductor material 155 that can be used in the present invention may be CdTe, CZT, T1Br or the like. The detectors 152, 153 and 154 employing the semiconductor material 155 can detect radiation using a power pulse without adding any optical sensor. A power pulse signal of each of the detectors 152, 153 and 154 is applied to and processed in the signal processing unit 157. The processed signal is then applied to the image processing unit 158 and converted into an image.

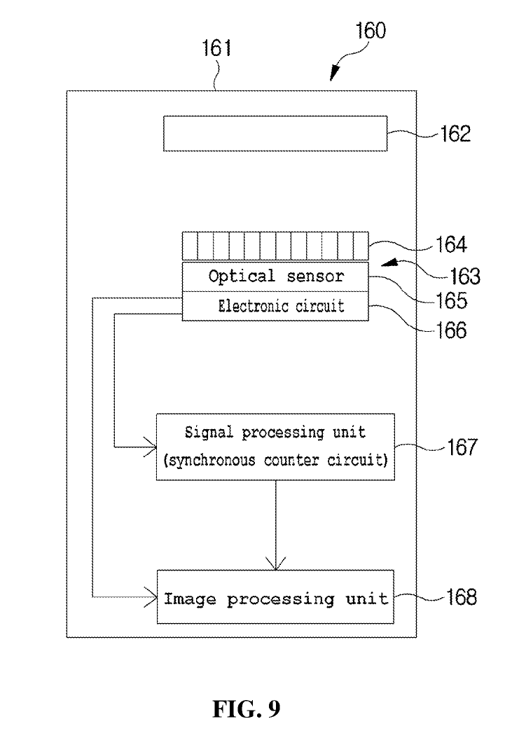

A three-dimensional scattered radiation imaging apparatus 160 shown in FIG. 9 includes a radiation detection unit 161 that detects radiation by a mechanical collimation method. The radiation detection unit 161 includes a collimator 162, a detector 163, a signal processing unit 167, and an image processing unit 168. The collimator 162 is a means that detects radiation having desired directionality in such a manner as to geometrically limit the radiation, and various kinds of collimators can be used depending on the detection site and purpose. The collimator 162 that can be used in the present invention may be a parallel-hole collimator, a pinhole collimator, URA, MURA, HURA or the like. The detector 163 detects the position and energy of radiation scattered from a radiation irradiated subject such as the patient P. The detector 163 includes a scintillator 164, an optical sensor 165, and an electronic circuit 166. PSPMT, SiPM or the like may be used as the optical sensor 165. The signal processing unit 167 receives information on the positions and energy of the radiation from the detector 163 so as to calculate the position of the radiation source in such a manner as to reversely track the incident direction of the radiation. The image processing unit 168 receives information from the signal processing unit 167 and displays the information as an image.

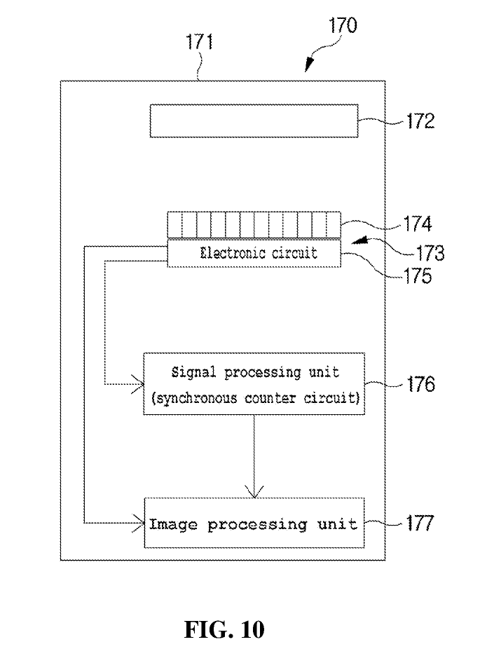

A three-dimensional scattered radiation imaging apparatus 170 shown in FIG. 10 includes another radiation detection unit 171 that detects radiation by a mechanical collimation method. The radiation detection unit 171 includes a collimator 172, a detector 173, a signal processing unit 176, and an image processing unit 168. The collimator 172 is a means that detects radiation having desired directionality in such a manner as to geometrically limit the radiation. The collimator 172 that can be used in the present invention may be a parallel-hole collimator, a pinhole collimator, URA, MURA, HURA or the like. The detector 173 includes a semiconductor material 174 and an electronic circuit 175. The signal processing unit 176 receives information on the positions and energy of the radiation from the detector 173 so as to calculate the position of the radiation source in such a manner as to reversely track the incident direction of the radiation. The image processing unit 177 receives information from the signal processing unit 176 and displays the information as an image.

Herein, the semiconductor material 174 that can be used in the present invention may be CdTe, CZT, T1Br or the like. The detector 173 employing the semiconductor material 174 can detect radiation using a power pulse without adding any optical sensor. A power pulse signal of the detector 173 is applied to and processed in the signal processing unit 176. The processed signal is then applied to the image processing unit 177 and converted into an image.

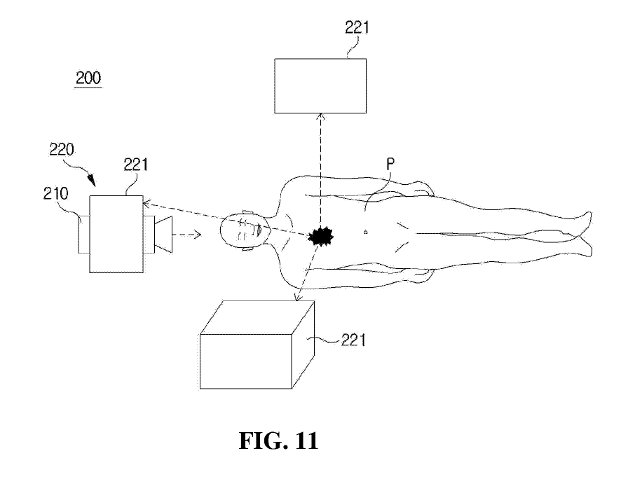

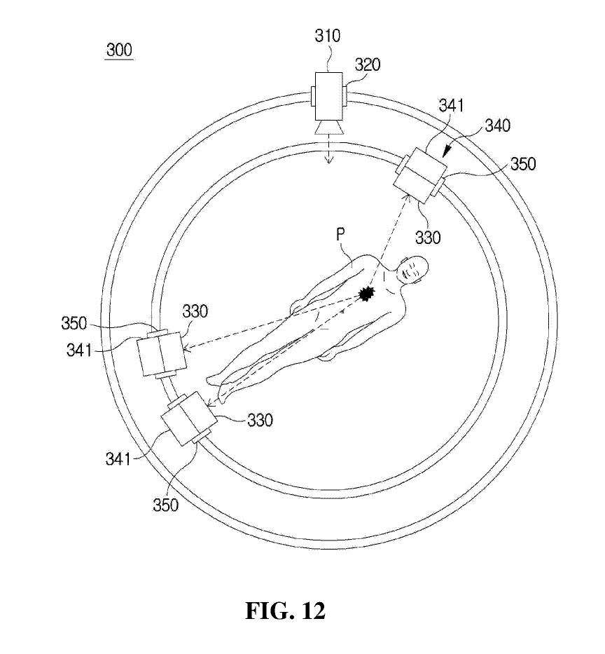

Meanwhile, FIGS. 11 and 12 are schematic diagrams showing various modifications of a radiological medical system having a three-dimensional scattered radiation imaging apparatus according to the present invention.

As shown in FIG. 11, a radiological medical system 200 including a three-dimensional scattered radiation imaging apparatus includes a radiation irradiation unit 210 that irradiates a subject (i.e., a patient P) with radiation, a three-dimensional scattered radiation imaging apparatus 220 that detects the irradiation position and the dose distribution of radiation, and a controller (not shown) that controls the radiation irradiation unit 210 and the three-dimensional scattered radiation imaging apparatus 220.

The three-dimensional scattered radiation imaging apparatus 220 includes a plurality of radiation detection units 221. One of the plurality of radiation detection units 221 is coupled to the radiation irradiation unit 210, and the remaining two radiation detection units 221 are disposed at different positions so as to be spaced apart from each other. The radiation detection unit 221 that can be used in the present invention may be implemented as either a Compton camera structure that employs the electronic collimation method as described above, or a mechanical collimation structure that employs the collimator. The plurality of the radiation detection units 221 may be mounted fixedly or mounted so as to be movable by a separate driver.

The three-dimensional scattered radiation imaging apparatus 220 of the radiological medical system 200 according to this embodiment can acquire image information of various directions by arranging, in various directions around the patient P, the plurality of the radiation detection units 221 capable of detecting radiation.

As shown in FIG. 12, a radiological medical system 300 including a three-dimensional scattered radiation imaging apparatus includes a radiation irradiation unit 310 that irradiates a subject (i.e., a patient P) with radiation, a irradiation unit driver 320 that movably operates the radiation irradiation unit 310 , a plurality of CT detectors 330, a three-dimensional scattered radiation imaging apparatus 340 that detects the irradiation position and the dose distribution of radiation, and a controller (not shown).

The radiation irradiation unit 310 is movably operated by the irradiation unit driver 320 so as to irradiate the patient P with radiation while rotating 360 degrees around the patient P. The radiation irradiation unit 310 and the irradiation unit driver 320 are controlled by the controller.

The three-dimensional scattered radiation imaging apparatus 340 includes a plurality of radiation detection units 341. The radiation detection units 341 that can be used in the present invention may be implemented as either a Compton camera structure that employs the electronic collimation method as described above, or a mechanical collimation structure that employs the collimator.

The plurality of radiation detection units 341 are respectively coupled to the plurality of CT detectors 330 and are respectively moved by a plurality of radiation detection unit drivers 350. Each of the plurality of radiation detection units 341 and each of the plurality of CT detectors 330 measure radiation scattered from the patient P in real time while rotating 360 degrees around the patient P by each of the detection unit drivers 350. The radiation detection unit 341 and the CT detector 330 that are coupled to each other can be operated cooperatively with each other to reconstruct a three-dimensional image.

The three-dimensional scattered radiation imaging apparatus 340 according to this embodiment is coupled to the CT detector 330 of a tomotherapy device so that the detection efficiency of radiation scattered from the patient P can be increased and the irradiation position and the dose distribution of radiation can be acquired in real time in a more accurate manner through fusion with a CT image containing excellent anatomical information.

As described above, the radiological medical system including the three-dimensional scattered radiation imaging apparatus according to the present invention can be configured in various manners within a range of achieving a structure capable of measuring radiation in real time during radiotherapy without being accompanied by an additional dose examination.

For example, the number of the radiation detection units constituting the three-dimensional scattered radiation imaging apparatus or the number of the detectors included in the radiation detection unit are not limited to the number illustrated in the drawings, but may be changed variously.

While the present invention has been described in connection with the exemplary embodiments illustrated in the drawings, they are merely illustrative and the invention is not limited to these embodiments. It will be appreciated by a person having an ordinary skill in the art that various equivalent modifications and variations of the embodiments can be made without departing from the spirit and scope of the present invention. Therefore, the true technical scope of the present invention should be defined by the technical spirit of the appended claims.

1. A three-dimensional scattered radiation imaging apparatus comprising:
a detection unit which includes a first detector for detecting the position and energy of radiation irradiated from a radiation source and scattered from a subject, a second detector for detecting the position and energy of radiation scattered from the first detector, and a third detector for detecting the position and energy of radiation scattered from the second detector;
a signal processing unit for receiving, from the first detector, the second detector, and the third detector of the detection unit, information on the positions and energy of the radiation detected by the first detector, the second detector, and the third detector of the detection unit so as to obtain the position of the radiation source in such a manner as to reversely track the incident direction of the radiation; and
an image processing unit for receiving information from the signal processing unit and displaying the information as an image.

2. The three-dimensional scattered radiation imaging apparatus according to claim 1, wherein the detection unit is implemented as a Compton camera structure in which each of the first detector, the second detector, and the third detector comprises a scintillator and an optical sensor.

3. The three-dimensional scattered radiation imaging apparatus according to claim 1, wherein the detection unit is implemented as a Compton camera structure in which each of the first detector, the second detector, and the third detector comprises a semiconductor material selected from among CdTe, CZT, and T1Br.

4. The three-dimensional scattered radiation imaging apparatus according to claim 1, wherein the signal processing unit calculates an energy E expressed as the following equation (3) through the following equations (1) and (2) when the direction of radiation irradiated from the radiation source to the subject is known, the energy E being absorbed by the subject:
$${hv}^{′} = \frac{hv}{1 + \frac{hv}{{m}_{0} {C}^{2}} ( 1 - cos θ )} \qquad ( 1 )$$
wherein hv′ denotes the energy of a photon scattered from the subject, hv denotes the energy of a photon irradiated from the radiation source, θ denotes the scattered angle of radiation scattered from the subject, and m₀c² denotes the rest mass of an electron,
E=hv−hv′ (2)
$$E = hv - {hv}^{′} = {hv}^{′} \frac{\frac{{hv}^{′}}{{m}_{0} {C}^{2}} ( 1 - cos θ )}{1 - \frac{{hv}^{′}}{{m}_{0} {C}^{2}} ( 1 - cos θ )} . \qquad ( 3 )$$

5. The three-dimensional scattered radiation imaging apparatus according to claim 1, wherein the signal processing unit detects the energy and direction of radiation scattered from the subject and incident on the first, second and third detects when the direction of radiation irradiated from the radiation source to the subject is not known, and calculates the dose of radiation irradiated to the subject by comparing a theoretical value and a computer simulation value/an actual measurement value.

6. The three-dimensional scattered radiation imaging apparatus according to claim 1, wherein the signal processing unit three-dimensionally represents the positions of radiation scattered from the first, second and third detectors, and represents them in a four-dimensional matrix, including the energy absorbed by the subject so as to calculate the energy absorbed by the subject.

7. The three-dimensional scattered radiation imaging apparatus according to claim 6, wherein the signal processing unit calibrates an absolute value for the energy absorbed by the subject through a simulation that is performed before the radiation irradiation when the angle of radiation incident on the subject is not known.

8. The three-dimensional scattered radiation imaging apparatus according to claim 1, wherein the detection unit is arranged in a direction where the degree of uncertainty of detection is low to fit the incident energy-dependent scattering distributions of radiation based on the Klein-Nishina formula depending on the energy absorbed by the subject and the energy scattered from the subject.

9. A radiation medical system comprising:
a radiation irradiation unit for irradiating a subject with radiation;
a radiation detection unit comprising a detection unit for detecting the position and energy of radiation irradiated from the radiation irradiation unit and scattered from a subject, a signal processing unit for receiving information on the positions and energy of the radiation detected by the detection unit from the detection unit so as to obtain the position of the radiation irradiation unit in such a manner as to reversely track the incident direction of the radiation, and an image processing unit for receiving information from the signal processing unit and displaying the information as an image; and
a controller for controlling the radiation irradiation unit and the radiation detection unit.

10. The radiological medical system according to claim 9, further comprising a driver for movably operating the radiation irradiation unit, and wherein the radiation detection unit is coupled with the radiation irradiation unit so as to detect radiation scattered from the subject while being movably operated together with the radiation irradiation unit by the driver.

11. The radiological medical system according to claim 9, further comprising:
an irradiation unit driver for movably operating the radiation irradiation unit; and
a detection unit driver for movably operating the radiation detection unit.

12. The radiological medical system according to claim 9, wherein the radiation detection unit is provided in plural numbers so as to be spaced apart from one another.

13. The radiological medical system according to claim 9, wherein the detection unit of the radiation detection unit comprises:
a first detector for detecting the position and energy of radiation irradiated from the radiation irradiation unitand scattered from the subject;
a second detector for detecting the position and energy of radiation scattered from the first detector; and
a third detector for detecting the position and energy of radiation scattered from the second detector.

14. The radiological medical system according to claim 9, wherein the radiation detection unit comprises a Compton camera structure in which the detection unit comprises a scintillator and an optical sensor.

15. The radiological medical system according to claim 9, wherein the radiation detection unit comprises a Compton camera structure in which the detection unit comprises a semiconductor material selected from among CdTe, CZT, and T1Br.

16. The radiological medical system according to claim 9, wherein the radiation detection unit further comprises a collimator for collimating radiation scattered from the subject and sending the collimated radiation to the detection unit.

17. The radiological medical system according to claim 9, further comprising a CT detector coupled with the radiation detection unit for reconstructing a three-dimensional image.

18. The radiological medical system according to claim 9, wherein the detection unit of the radiation detection unit is arranged in a direction where the degree of uncertainty of detection is low to fit the incident energy-dependent scattering distributions of radiation based on the Klein-Nishina formula depending on the energy E absorbed by the subject and the energy scattered from the subject.

19. A method for arranging a three-dimensional scattered radiation imaging apparatus, comprising the steps of:
(a) provisionally arranging the three-dimensional scattered radiation imaging apparatus comprising a detection unit of a Compton camera structure for detecting the position and energy of radiation irradiated from a radiation source and scattered from a subject, a signal processing unit for receiving information on the positions and energy of the radiation detected by the detection unit from the detection unit so as to obtain the position of the radiation source in such a manner as to reversely track the incident direction of the radiation, and an image processing unit for receiving information from the signal processing unit and displaying the information as an image;
(b) calculating an energy E expressed as the following equation (3) through the following equations (1) and (2) using the detection unit of the three-dimensional scattered radiation imaging apparatus, the energy E being absorbed by the subject:
$${hv}^{′} = \frac{hv}{1 + \frac{hv}{{m}_{0} {C}^{2}} ( 1 - cos θ )} \qquad ( 1 )$$
wherein hv′ denotes the energy of a photon scattered from the subject, hv denotes the energy of a photon irradiated from the radiation source, θ denotes the scattered angle of radiation scattered from the subject, and m₀c² denotes the rest mass of an electron.
$$\begin{matrix}E = hv - {hv}^{′} & ( 2 ) \\ E = hv - {hv}^{′} = {hv}^{′} \frac{\frac{{hv}^{′}}{{m}_{0} {C}^{2}} ( 1 - cos θ )}{1 - \frac{{hv}^{′}}{{m}_{0} {C}^{2}} ( 1 - cos θ )} ; & ( 3 )\end{matrix}$$
(c)_calculating an average value of the energy-dependent ratios of a total attenuation coefficient (the probability of reaction and attenuation of radiation per unit length) and a scattering coefficient (the probability of Compton scattering of radiation per unit length) of radiation based on the amount of incident radiation in the energy, and calibrating the energy E of the photon absorbed by the subject and the energy of the photon scattered from the subject, which are calculated in step (b) based on the averaged ratios; and
(d) adjusting the position of the three-dimensional scattered radiation imaging apparatus in a direction where the degree of uncertainty of detection is low to fit the incident energy-dependent scattering distributions of radiation based on the Klein-Nishina formula depending on the energy E absorbed by the subject and the energy scattered from the subject, which are calibrated in step (c)

20. The method according to claim 19, wherein steps (b) to (d) are performed repeatedly in order to further optimize the arrangement of the detection unit of the three-dimensional scattered radiation imaging apparatus.
